(19) United States
(12) Patent Application Publication
(10) Pub. No.: US 2026/0021318 A1
(43) Pub. Date: Jan. 22, 2026

(54) SYSTEM AND METHOD OF HIGH FREQUENCY NEUROMODULATION FOR TRANSCRANIAL MAGNETIC STIMULATION

(71) Applicant: VIRGINIA COMMONWEALTH UNIVERSITY, Richmond, VA (US)

(72) Inventors: Ivan C. CARMONA-TORTOLERO, Richmond, VA (US); Ravi L. HADIMANI, Richmond, VA (US); Mark S. BARON, Richmond, VA (US); Deepak KUMBHARE, Richmond, VA (US)

(21) Appl. No.: 18/854,813

(22) PCT Filed: Apr. 26, 2023
(86) PCT No.: PCT/US2023/066255

§ 371 Date: Oct. 7, 2024

Related U.S. Application Data
(60) Provisional application No. 63/334,767, filed on Apr. 26, 2022.

(51) Int. Cl. A61N 2/02; A61N 2/00

(52) U.S. Cl. CPC A61N 2/02; A61N 2/006

(57) ABSTRACT

Examples include spatially positioning, in a three-dimensional (3D) space, a conductive coil and a living subject in a 3D spatial relation in which a target 3D region of the living subject's tissue is within a designated 3D electric field formation region for the conductive coil. An energizing source feeds a low frequency (LF) modulated high frequency (HF) carrier voltage to terminals of the conductive coil. This urges a corresponding LF modulated HF coil current, of maximum magnitude MA, through the conductive coil. The HF frequency of the HF coil current produces a magnetic flux with a corresponding HF related rate of change. Optionally, a secondary coil feeds an unmodulated HF signal that spatially overlaps the modulated HF signals.

### CROSS-REFERENCE TO RELATED APPLICATIONS

This application claims priority to U.S. Provisional Application 63/334,767 filed Apr. 26, 2022, which is hereby incorporated by reference in its entirety.

### TECHNICAL FIELD

This invention generally relates to transcranial magnetic stimulation and, more particularly, to coil sourced, modulated high frequency transcranial magnetic stimulation.

### BACKGROUND

Transcranial magnetic stimulation (TMS) is a technique for producing within target three-dimensional (3D) regions of a living subject's neural tissue, an electric field having strength sufficient to cause a depolarization of neural membranes significant enough to induce neural spikes. Control of the electric field strength in turn controls the inducement of neural spikes, and is therefore a regulation of the synaptic activity of neurons in the 3D target region, in terms of firing rate and patterns. The term “magnetic” is included in the technique's name because TMS establishes the electric field in the target region by passing a time varying electric current through a particularly structured and arranged conductive coil . . . a time-varying magnetic flux density. . . .

Current TMS technology is restricted to frequencies in which neurons have demonstrated to respond under electrical/electromagnetic stimulation, typically in a range from 0 to 2.5 kHz, sometimes up to 3 kH.

Desirability of high frequency fields for neuromodulation has been known. Present TMS technology operates within a limited range of frequency because, typically, neurons are not able to respond to stimulation frequencies beyond 3 kHz. This limitation results from the neuron membrane having a membrane time constant possessing a low pass attenuates the response to the E-field in such a manner that the ionic currents are not able to increase the internal potential inside the soma at high frequencies. Therefore, an action potential cannot be triggered to produce the neural spike. This represents the main reason why high frequency (out of the TMS band) has not been used to increase the −dB/dt so far.

### SUMMARY

Exemplary benefits include requirement of less flux density than convention techniques, and therefore requirement of less coil current. This is a secondary benefit of providing, in regions of interest within a subject tissue, a faster changing flux density, i.e., larger magnitude derivative of flux density with respect to time, and therefore not requiring as high of a flux density as needed for conventional low frequency oscillating coil current TMS devices.

Other exemplary benefits include reduction of |B| and, therefore, of the necessary current in the TMS coils to produce the required |E|.

Another exemplary benefit is lower coil current which, in turn, obtains a reduction in resistive power loss. Reduction of the power dissipation in TMS coils as a function of the reduced TMS currents. Reduced power consumption can mean less heat. This in turn can make possible a reduced restriction in repetitive TMS (rTMS) because of the reduce power dissipation in coils.

Another exemplary benefit is reduction of the size of the existing TMS coils (r).

Another exemplary benefit is an increase of focality and penetration depth through smaller coils.

Another exemplary benefit is noise-reduced, effectively noiseless TMS equipment and therapies.

Other exemplary benefits include hardware reduction in the power electronic requirements compared to existing technology, and reduction in the size of the equipment and increase of portability.

This Summary identifies example features and aspects and is not an exclusive or exhaustive description of disclosed subject matter. Whether features or aspects are included in or omitted from this Summary is not intended as indicative of relative importance of such features or aspects. Additional features are described, explicitly and implicitly, as will be understood by persons of skill in the pertinent arts upon reading the following detailed description and viewing the drawings, which form a part thereof.

### BRIEF DESCRIPTION OF THE DRAWINGS

FIG. 1A shows a two-dimensional (2D) projection of an example arrangement of a coil transmission, modulated high frequency (HF) electromagnetic (EM) field, transcranial magnetic stimulation (TMS) system according to one or more embodiments.

FIG. 1B shows FIG. 1A overlaid with a graphic representation of applied modulated HF coil voltage, coil current, time-varying magnetic flux and stimulation electric field.

FIG. 2A shows a simulation generated amplitude versus time plot of one example HF EM carrier wave signal for use in generating a modulated HF EM fields for various TMS systems and methods according to one or more embodiments.

FIG. 2B shows a simulation generated amplitude versus time plot of a low frequency (LF) modulating signal for coil transmission of modulated HF EM fields for various TMS systems and methods according to one or more embodiments.

FIG. 2C shows a simulation generated amplitude versus time plot of a LF amplitude modulated (AM) HF voltage for coil transmission of modulated HF EM fields for various TMS systems and methods according to one or more embodiments.

FIG. 3A shows a frequency spectrum for the HF carrier signal.

FIG. 3B shows a frequency spectrum for the LF stimulation signal.

FIG. 3C shows a frequency spectrum for the AM modulated HF stimulation signal.

FIG. 4 shows a partially simplified graphic model of aspects of a neural structure in an example extracellular environment, labeled to show example ionic currents.

FIG. 5 shows an enlarged scale graphic model of a portion of a neuron membrane region of the neural structure modelled in FIG. 4, annotated to show example Na+ ion motion through the membrane.

FIG. 6 shows by an equivalent circuit diagram, a modelled envelope-detection behavior with rectifier that can, in one or more embodiments, be contributed by the neuron membrane in accordance with the FIG. 5 model.

FIG. 7 shows an example simulation-generated plot of voltage versus time, and voltage magnitude versus frequency spectrum of an example modulated feed voltage in a simulated operation of an example coil transmission, modulated HF EM field TMS system and method according to one or more embodiments.

FIG. 8 shown one example simulation-generated plot of coil current versus time, and of coil current magnitude versus frequency in an example coil transmission, modulated HF EM field TMS system and method according to one or more embodiments.

FIG. 9 shows an example simulation-generated plot of magnetic flux density versus time, and flux magnitude versus frequency spectrum in a targeted plane in an example coil transmission, modulated HF EM field TMS system and method according to one or more embodiments.

FIG. 10 shows an example simulation-generated plot of target region electric field versus time, and target region electric field magnitude versus frequency spectrum in an example coil transmission, modulated HF EM field TMS system and method according to one or more embodiments.

FIG. 11 shows an example simulation-generated plot of electric field versus time and the frequency spectrum of magnitudes of the e-field seen by neurons in the target region of a neuron tissue in an example coil transmission, modulated HF EM field TMS system and method according to one or more embodiments.

FIG. 12 shows an example simulation-generated gain versus frequency plot representing a low pass filter characteristic of the frequency response of neurons.

FIG. 13 shows by an equivalent circuit diagram a feature of a bias that theoretical sampling-and-hold behavior, with no rectifier, for optional adaptation directed to neuron detection of the low-frequency envelope from an asymmetric, circuit-rectified AM signal.

FIG. 14 shows the equivalent circuit of FIG. 13, with two mutually overlapping induced (modulated and non-modulated) E-fields, for induced demodulation via constructive temporal interference over a brain tissue.

FIG. 15 shows a 2D projection of an HF demodulation carrier coil supplemented version of the FIG. 1A coil transmission, modulated HF TMS system according to one or more embodiments.

FIG. 16 shows a schematic of an example circuit for generating the modulated HF voltage for an modulated HF EM field TMS system according to one or more embodiments.

FIG. 17 shows a schematic of an example circuit for generating the differentiated FM modulated HF voltage for modulated HF EM field TMS systems and methods according to one or more embodiments.

FIG. 18 shows a simulated time domain plot and spectral plot of an intermediate FM signal generated by a component of the FIG. 17 differentiated FM modulated HF voltage generator.

FIG. 19 shows a simulated time domain plot and spectral plot of a differentiated FM signal output of the FIG. 17 differentiated FM modulated HF voltage generator.

FIG. 20 shows an H-bridge implementation for the differentiated FM modulated HF voltage.

### DETAILED DESCRIPTION

Current techniques for transcranial magnetic stimulation operate in a relatively limited range of frequencies, usually up to 3 kHz. The limitation is mainly imposed by the typical physiological response of neurons to time-varying E-fields and current densities induced through conventional techniques.

Irrespective of any theoretical restriction for the frequency of an electromagnetic pulse to produce an interaction with the ionic species of the extracellular environment of neurons, for a neuron to be induced to fire, the duration of the stimulating pulse should be long enough to allow the membrane potential to reach the depolarization threshold. A result is that low frequencies—whose periods are longer—can give the neuron sufficient time to reach the threshold. Smaller periods of high frequencies may appear to not permit this to occur. This aspect appears to reduce the frequency requirements to the minimum possible.

Despite the heretofore mentioned, a counter reason motivates designers make the frequency in TMS as high as possible. The reason arises from Faraday's Law of induction, which dictate that the electric field obtained from the variation in time of an applied magnetic field is a direct function of the time derivative of its magnetic flux density (B). This implies that the higher the frequency of B, the higher the resulting time derivative and, therefore, the E-field magnitude. Consequently, the definition of a suitable frequency of operation in TMS is a tradeoff between a low value that makes the membrane depolarization possible and a reasonably high value for an adequate E-field strength. An appropriate value of frequency of TMS pulses is typically between 2 kHz and 3 kHz in practice.

It appears therefore that the use of high frequency is not possible with the current conception of TMS technology, likely because the operation frequency is perceived as a parameter that must meet the physiological requirements for neuron stimulation, instead of being conceptualized as a means for energy transfer.

However, some efforts have been directed to the introduction of high frequency to some neuromodulation methods. One such effort. often referred to as “temporal interference” (TI), is a technique in which high-frequency components are applied through electrodes to obtain a superposition of signals inside the brain tissue. The temporal interference produces an envelope modulation, a type of modulation that generates a low-frequency component in the envelope of the resulting signal that neurons can detect.

An additional problem related to the frequency range of conventional TMS technique MS frequency is that sound levels can be sensed by some as overly loud.

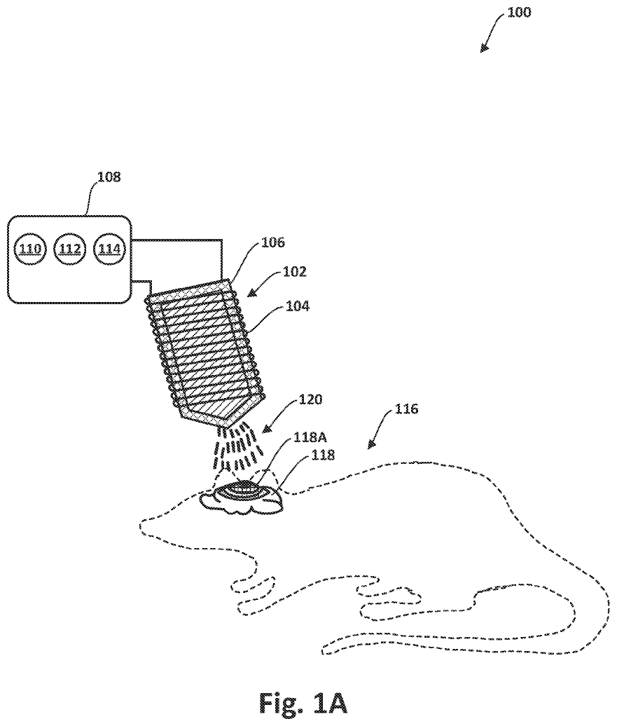

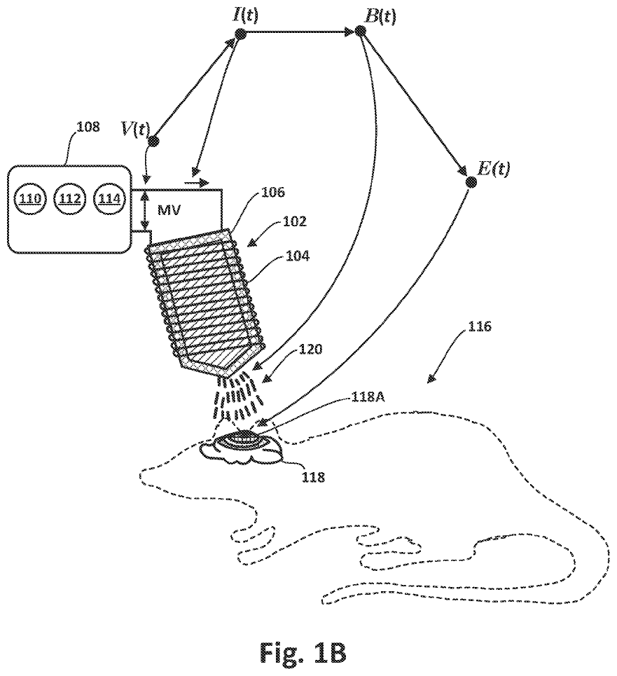

FIG. 1A shows a two-dimensional (2D) projection of an example arrangement of a coil transmission, modulated high frequency (HF) electromagnetic (EM) field, transcranial magnetic stimulation (TMS) system 100 according to one or more embodiments. The system 100 includes a conductive coil 102 formed of a conductive winding 104 around a ferromagnetic core 106. The conductive winding has two terminals each connecting to a modulated HF neurostimulator voltage source 108. The modulated HF neurostimulator voltage source 108 can include an amplitude control 110, 1 frequency control 112, and a modulation index control 114.

For purposes of illustration a laboratory rat 116 is shown supported, e.g., resting on a surface in a position wherein the head and therefore the corresponding brain 118 is under a lower tip of the conductive coil 102. Activating the modulated HF neurostimulator voltage source 108 applies a corresponding modulated HF neurostimulator voltage to the terminals of the conductive coil 102. This in turn urges a modulated HF current through the conductive coil 102, causing a time varying flux density 120 to pass into a region of the brain 118. The time varying flux density 120 in turn creates a TMS stimulation electric field 118A in the brain 118.

FIG. 1B shows FIG. 1A overlaid with a graphic representation of applied modulated HF coil voltage, coil current, time-varying magnetic flux and stimulation electric field.

Regrading structure of the conductive coil 102, a non-limiting example, which was simulated uses a 20-turn, 4-layer coil with a height of 10 mm and an outer diameter of 15 mm. The core material used was a cylinder of AISI 1010 steel with a diameter of 3 mm and a height of 10 mm. The stimulation tone used was 1.5 kHz. The carrier frequency used was 25.5 kHz.

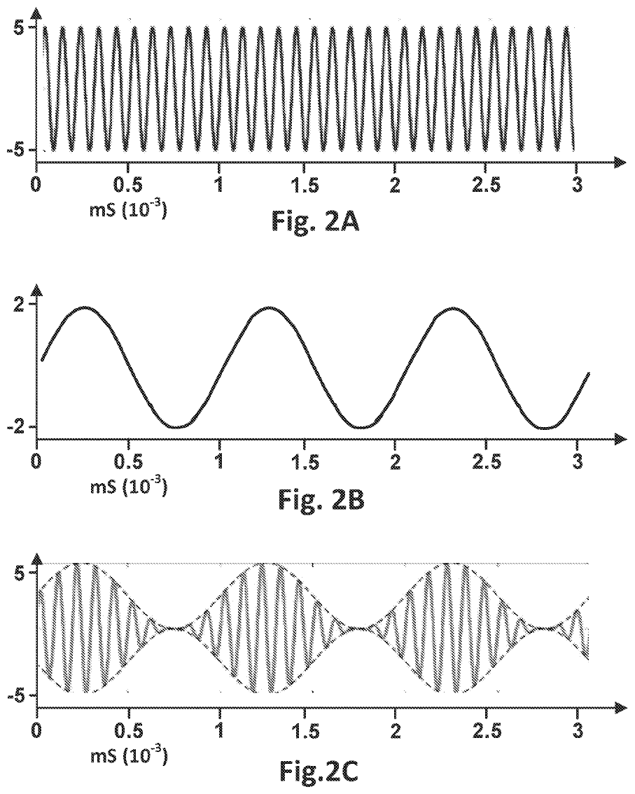

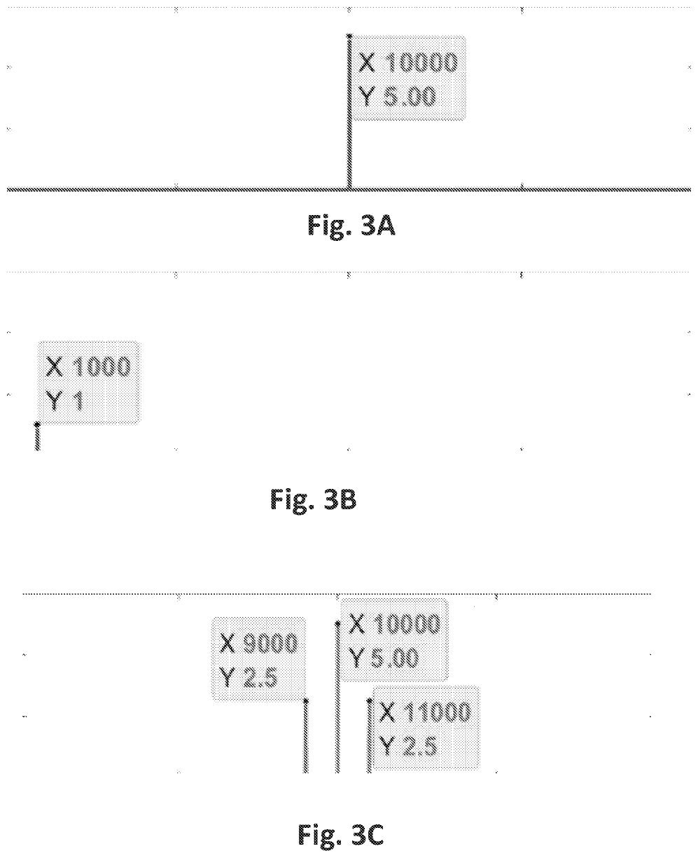

FIG. 2A shows a simulation generated amplitude versus time plot of one example HF EM carrier wave signal that can be generated within the modulated HF neurostimulator voltage source 108. FIG. 2B shows a simulation generated amplitude versus time plot of an LF modulating signal and FIG. 2C shows a simulation generated amplitude versus time plot of a LF AM HF voltage for coil transmission of modulated HF EM fields for various TMS systems and methods according to one or more embodiments. The FIGS. 2B and 2C may also be generated within the modulated HF neurostimulator voltage source 108. FIG. 3A shows a frequency spectrum for the HF carrier signal. FIG. 3B shows a frequency spectrum for the LF stimulation signal. FIG. 3C shows a frequency spectrum for the AM modulated HF stimulation signal.

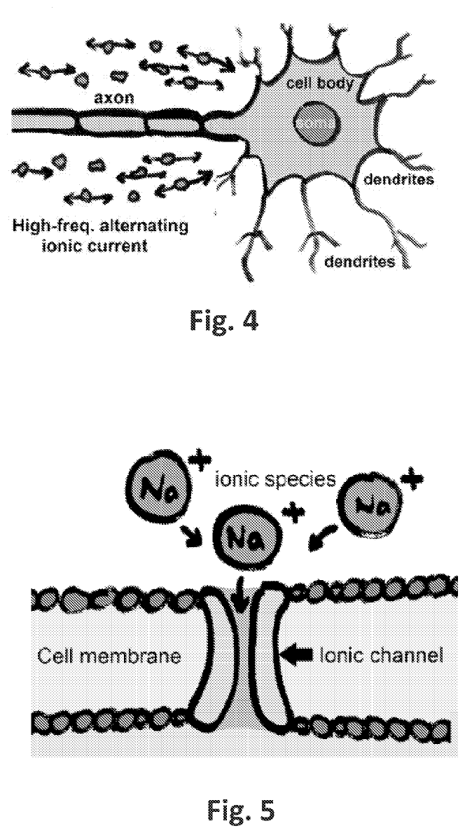

FIG. 4 shows a partially simplified graphic model of aspects of a neural structure in an example extracellular environment, labeled to show example ionic currents.

FIG. 5 shows an enlarged scale graphic model of a portion of a neuron membrane region of the neural structure modelled in FIG. 4, annotated to show example Na+ ion motion through the membrane.

Going back to the waveform of the magnetic flux density (B), assume that the operating frequency is intentionally selected to be out of the TMS range (i.e., 3 kHz), in a frequency tone that we will call carrier frequency, “fc”. This will automatically make the induced E-field ineffective to stimulate the neurons. Now, assume that we can multiply this carrier tone by a different tone of unitary amplitude, and frequency within the TMS range (i.e., 3 kHz). We will call this second component “message frequency” (fm) or “stimulating tone” Expression (6) contains this product as shown next.

B(t)=[Am·cos(2π·f m·t)]×[Ac·cos(2π·f c·t)].
where Ac is the amplitude is the carrier tone. For the example represented in FIGS. 2A, 2B, 2C, 3A, 3B, 3C: fm=1 kHz, fm=10 kHz, Am=1 and Ac=5.

Now, we say that the amplitude of the high-frequency carrier tone is modulated by the low-frequency stimulating tone. This means that the envelope formed by the peaks of the resulting high-frequency product signal will vary following the waveform of the stimulating tone.

In other words, the amplitude-modulated signal implicitly contains the waveform of the stimulated tone (fm), in a version of higher frequency (fc), meaning that the stimulating tone has been shifted in frequency. observed the frequency shifting of the AM signal obtained with the Fast Fourier Transform (FFT), showing two sidebands (single tones shown as deltas), located at fc−fm and fc+fm (9 kHz and 11 kHz, respectively for this example). An additional tone at the carrier frequency fc (10 kHz) is also observed as part of the AM modulation process, to provide the signal with more power.

At this point, we can observe through the amplitudes of the deltas, how the power/energy content (depending on whether the pulse is repetitive or not) of an amplitude-modulated signal is shifted in frequency to occupy a bandwidth of BW=2□fm. This will occur over a frequency band between fc−fm and fc+fm, which clearly shows that, if the AM signal represented the induced E-field with TMS, it would be applied as an out-of-band/modulated signal to the brain tissue, which would not directly stimulate the neurons. However, it would contain the original stimulating tone and energy to be recovered back to the baseband through different methods to be tested.

Because of the lack of reference in the literature about this specific novel topic, we provided three means for the neurons would respond to the presence of an amplitude-modulated E-field. For this, we have theorized three (3) possible response mechanisms, each of which leads to a different method for the demodulation and recovery of the stimulating tone over the brain tissue. Hence, during the design process, we needed to provide the stimulator with the ability to operate under all three scenarios.

FIG. 4 shows a partially simplified graphic model of aspects of a neural structure and of an example extracellular environment, with markings showing example ionic currents.

FIG. 5 shows an enlarged scale graphic model of a representative portion of a neuron membrane region of the neural structure modelled in FIG. 4, with marking showing example Na+ ion motion through the membrane.

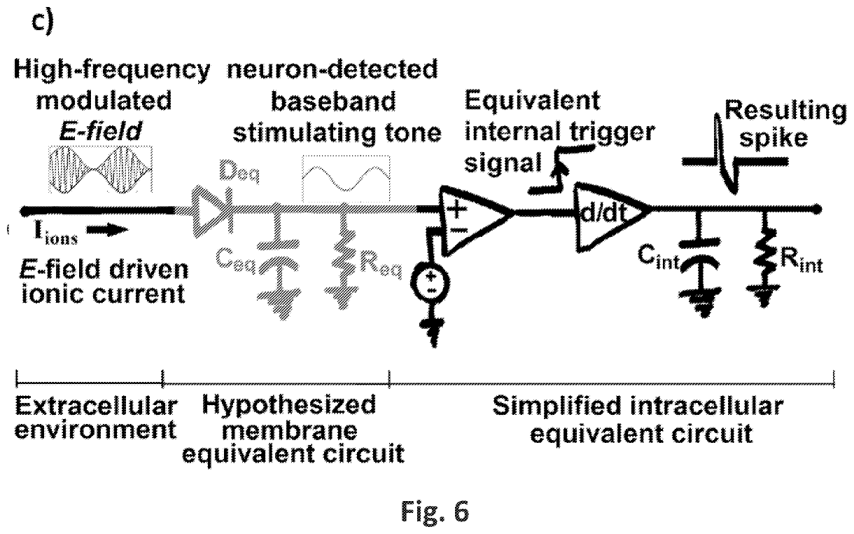

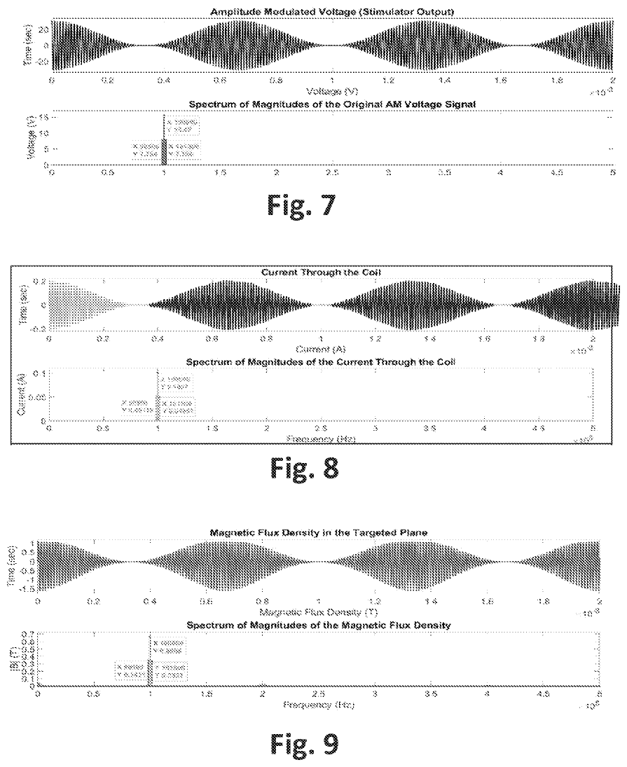

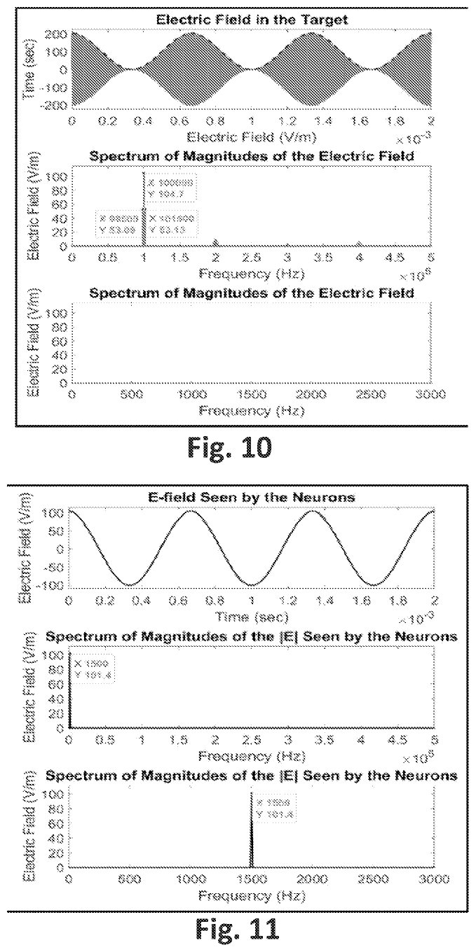

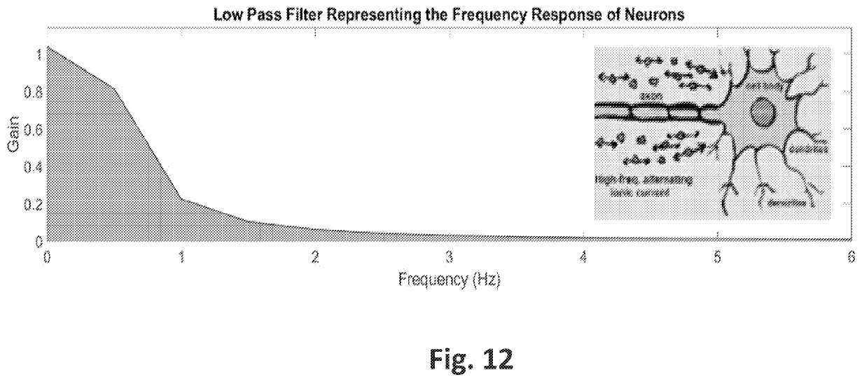

FIG. 6 shows an example equivalent circuit diagram of a modelled envelope-detection behavior with rectifier that can, in one or more embodiments, be contributed by the neuron membrane in accordance with the FIG. 5 model.

Auto-demodulation based on the natural envelope-detection behavior of the neuron membrane.

The first sub-hypothesis of the neural response to the AM/DSM E-field states that, although the symmetry between the upper and lower envelopes, neurons would respond to only one of them, acting as a voltage follower with a rectifier diode.

Rationale: We depart considering the voltage-triggered ionic channels in the neuronal membrane and the inertial characteristic of the ionic species, whose mass would prevent them from being suddenly accelerated/deaccelerated at high frequencies. These are two possible causes for the ionic currents to flow in just one direction at high frequency. Based on this, we believe that neurons could exhibit the behavior of a rectifier to the high-frequency amplitude modulated waveform of the E-field around them. This behavior, in addition to the low-pass filter characteristic of the neural membrane, would make neurons act as a natural envelope-detector circuit that would auto-demodulate any amplitude modulated signal, recovering the stimulating envelope within the TMS frequency range.

Embodiments of the disclosure provide a novel neuromodulator equipment that uses—for the first time to our knowledge—modulation techniques (AM/DSB-SC, ASK and FM) at high frequency (tens of kHz) for transcranial magnetic stimulation (TMS). The method modulates a high-frequency carrier (Xc)—located outside of the commercial TMSfrequency range—a low-frequency stimulation signal (Xs)—located within the stimulable range for neurons—over to generate afrequency shifling that takes the stimulating energy out of the stimulable baseband. This aims to exploit the capabilities of operating non-invasive TMS coils with elevated −dB/dt and frequency.

In an embodiment, an apparatus can include a coil—mutual support—connected to an LF modulated HF carrier coil voltage driver. drive HF or an arrangement having an arrangement comprising a plurality of primary coils, in combination with a secondary coil or an arrangement of a plurality of secondary coils. I

In one or more embodiments, an active E-field envelope recovery device can recover, or assist in recovering, the stimulation energy inside the brain volume and bring the signal back to the stimulatable range. In an aspect E-field induced by a non-modulated carrier over a secondary coil is used to overlap the modulated E-field induced by the primary coil.

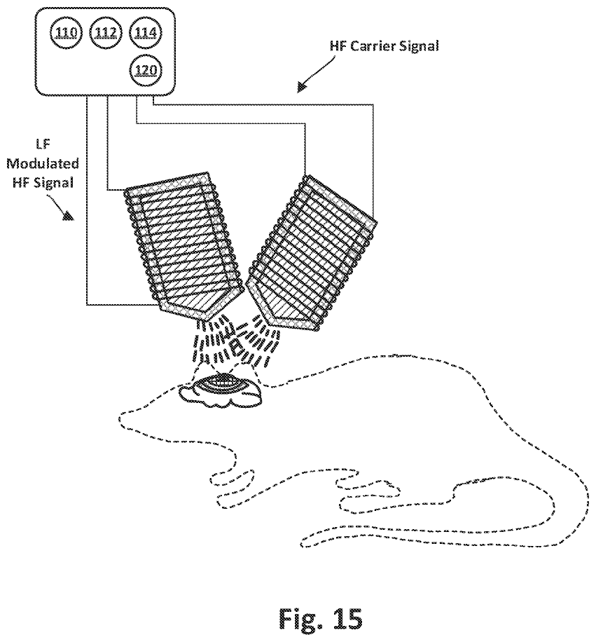

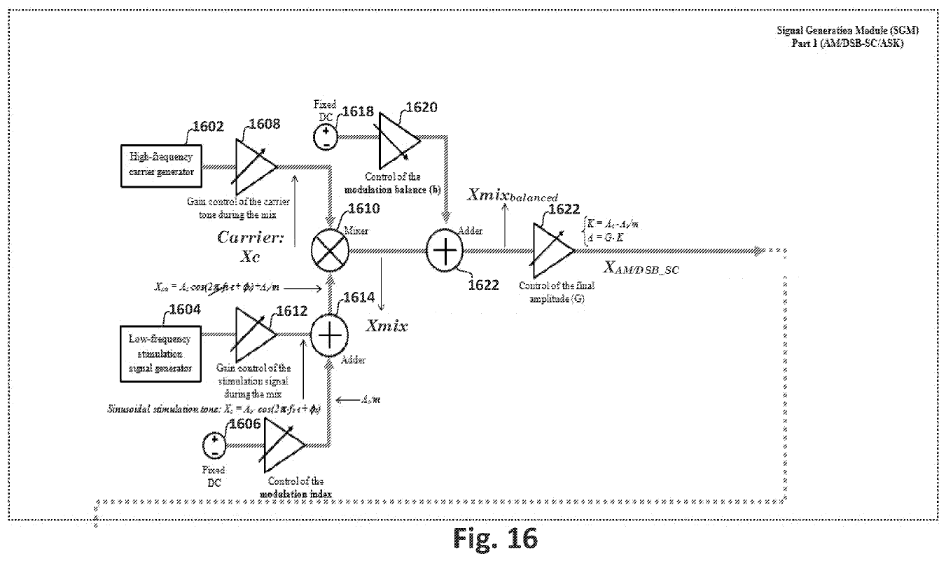

An example is shown in FIG. 15 shows a 2D projection of an HF demodulation carrier coil supplemented version of the FIG. 1A coil transmission, modulated HF TMS system according to one or more embodiments.

In the case of the AM (amplitude modulation) and DSB-SC (double-side band with suppressed carrier) modulations, when they are in phase and vectorially aligned, the superposition of both E-fields results in a doubly shifted frequency spectrum of the modulated signal with components around twice the carrier frequency.

$$2 \cdot {f}_{c} - {BW}_{{X}_{s}} \qquad Equation ( 1 )$$

$$2 \cdot {f}_{c} + {BW}_{{X}_{s}} \qquad Equation ( 2 )$$

and around zero [−BW_{X}_{ₛ}, BW_{X}_{ₛ},]. This last component represents the baseband of the original stimulating tone that will be recovered from the envelope of the modulated signal thanks to the low pass behavior of the neuron membrane.

The AM and DSB-SC methods just differ in that the AM will result in an additional DC component in the demodulated signal, whereas the DSB-SC does not produce any DC level because of its suppressed carrier. For some applications, DSB-SC can be preferable as it can provide various benefits, e.g., concentration of power in the stimulating side bands as opposed to the carrier. The latter does not necessarily provide direct stimulation of neurons. However for flexibility purposes, the system has been though to allow a continuous change in the modulation index (m) from 0 to infinity. This can be obtained by the control of the gain in the carrier added to the product of the stimulating tone (message) and the same carrier. This will allow to have a precise control on the final TMS current, B-field and E-field magnitude of the modulated signal and, therefore, of the stimulation component in baseband.

In an embodiment, there is another particular case of the AM/DSB-SC in which the stimulation signal is not a single sinusoidal tone, but a train of pulses. Although it could be a square signal, the stimulating train of pulses could be something as complex as a protocol of several pulses grouped in bursts with a certain pulse width, inter-pulse period, inter-burst period and a number of bursts defined or undefined (continuous mode).

The case with the frequency modulation (FM) is a particular one in which a broadband frequency modulated signal is produced first using the same low frequency stimulating tone and carrier that would be used in the AM or DSB-SC cases. In one or more embodiments, The generated FM signal is differentiated in the time domain, producing a dFM/dt signal. This new differentiated signal will be a frequency-modulated carrier modulated in amplitude at the same time by the stimulating tone. Then, this would be a FM-AM signal. From this point, the signal can be treated as any other AM or DSB-SC signal and the same previously mentioned demodulation method can be used.

In another one or more embodiments FSK neuromodulation method will be a particular case of the FM method. When the modulating signal is a square signal or a train of pulses as in ASK. This particular case will also allow the system to deliver train of pulses in bursts to follow a protocol.

The waveforms shown in the diagram are just a reference of the waveforms of current in each of the coils obtained after the delivery of the correspondent PWM signal. The actual waveforms will depend on the specific modulation method.

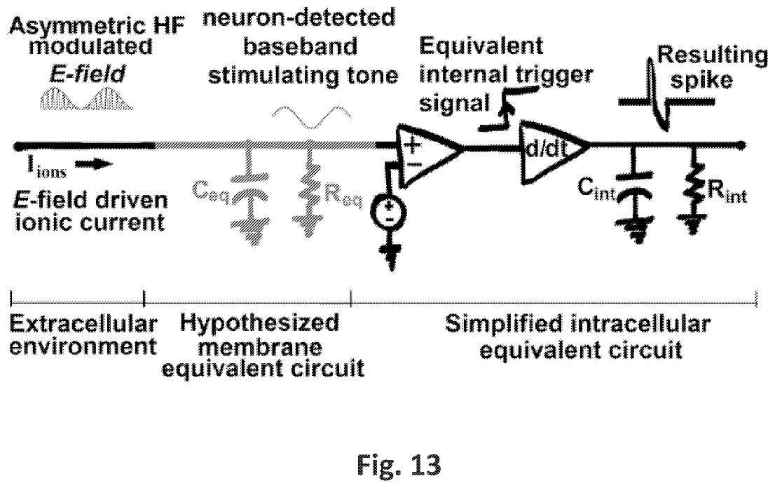

FIG. 13 shows by an equivalent circuit diagram a feature of a bias that theoretical sampling-and-hold behavior, with no rectifier, for optional adaptation directed to neuron detection of the low-frequency envelope from an asymmetric, circuit-rectified AM signal. This feature provides for cases where neurons may not exhibit a rectifier action but, by adding a bias, can still provide a tracking of just one side of the envelope.

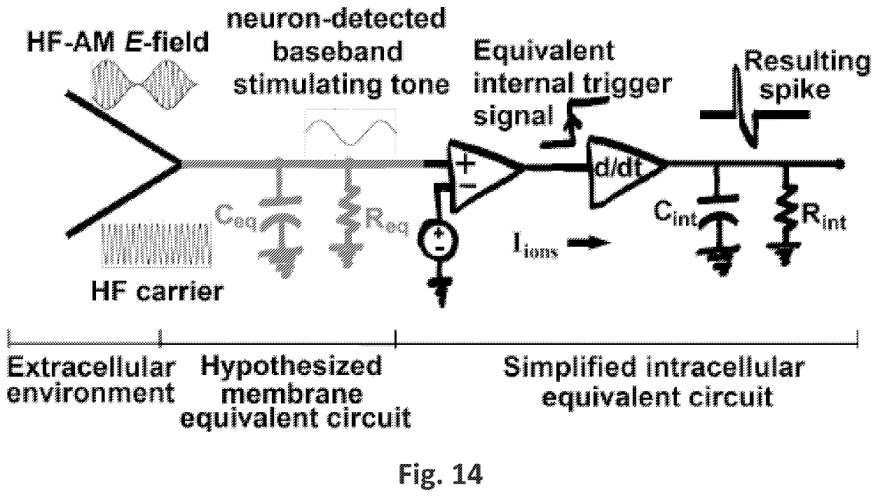

FIG. 14 shows by equivalent circuit another embodiment which provides a constructive temporal interference utilization of the HF electric field, by mutual temporal overlapping of a modulated HF E-field and a non-modulated HF E-field. for induced demodulation via constructive temporal interference over a brain tissue.

Neuromodulation with FM/FSK TMS Current Waveform:

In this embodiment, the neuromodulation method will not use exactly a frequency modulated signal, but the time derivative of it. This is the signal that is going to be delivered to the power electronic module (PEM) when switched from other neuromodulation modes.

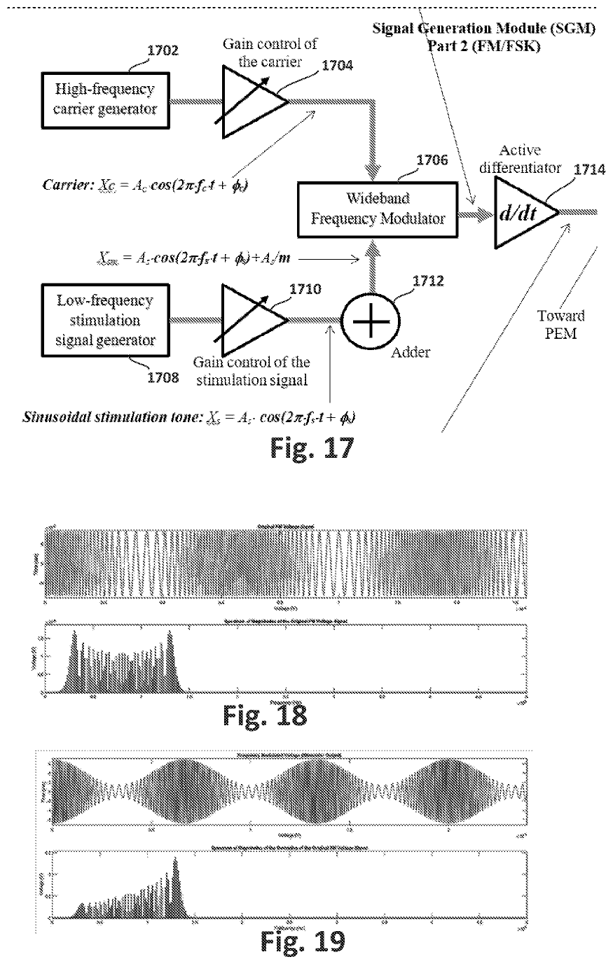

FIG. 17 shows a schematic of an example circuit for generating the differentiated FM modulated HF voltage for modulated HF EM field TMS systems and methods according to one or more embodiments.

FIG. 18 shows a simulated time domain plot and spectral plot of an intermediate FM signal generated by a component of the FIG. 17 differentiated FM modulated HF voltage generator.

FIG. 19 shows a simulated time domain plot and spectral plot of a differentiated FM signal output of the FIG. 17 differentiated FM modulated HF voltage generator.

The diagram in FIG. 17 shows an example on how to obtain the modulated signal. Notice that we are not being specific in the method used to obtain the first wideband frequency modulated signal, since this can be done by different existing means, including the use of integrated circuits (IC), and is a well-known topic in the electronics field. However, it is the use of the time derivative of this signal, and the underlying parts of the process, what introduces the novelty on this method.

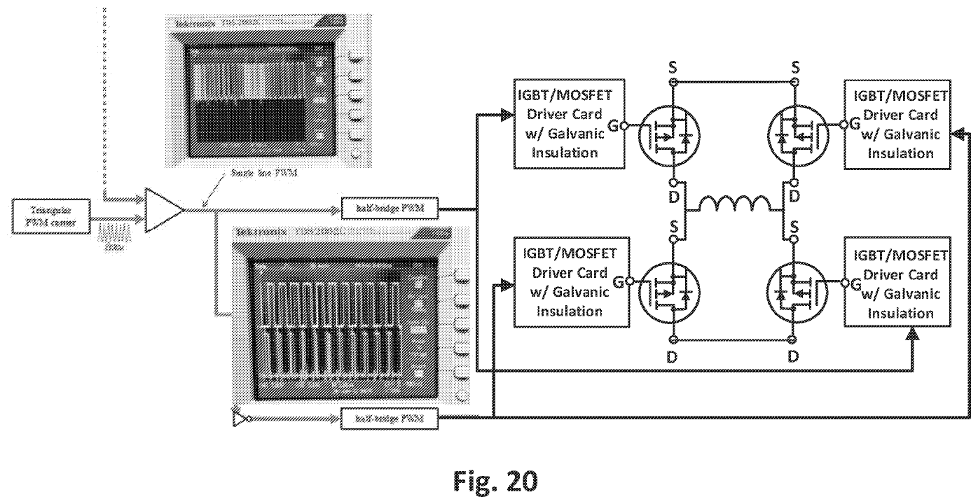

FIG. 20 shows an H-bridge implementation for the differentiated FM modulated HF voltage. Referring to FIG. 20, the modulated signal or a non-modulated carrier is given to the input to be converted in a PWM signal. Then, the signal is converted in a bipolar PWM using a H-bridge made of fast switches (MOSFETs in this case, but they could be IGBT or any other technology). The switches must be driven by external drivers with galvanic insulation from the control and signal generation side. The switching frequencies should be in the order of few to several kilohertz.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one, or both of the limits, ranges excluding either or both of those included limits are also included in the invention.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

We claim:

1. A reduced current method for establishing, within a tissue, a tissue simulating electric field, comprising:
spatially positioning, in a three-dimensional (3D) space, a conductive coil and a living subject into a cooperative 3D spatial relation wherein a target 3D region of the living subject's tissue is within a designated 3D electric field formation region for the conductive coil;
applying a low frequency (LF) modulated high frequency (HF) carrier voltage to terminals of the conductive coil, forcing a corresponding LF modulated HF coil current, of maximum magnitude MA, through the conductive coil, wherein:
the HF frequency is greater than approximately 8 KHz and the LF frequency is less than approximately 4 KHz, and
MA, the HF frequency, and a dimension and a geometry of the conductive coil are configured in combination to produce a time-changing magnetic flux density that establishes within the designated 3D electric field formation region an electric field having a magnitude that is at least approximately 100 volts per meter.

2. A reduced coil current method according to claim 1, wherein the LF modulation is a sinusoid and HF carrier is a sinusoid.

3. A reduced coil current method according to claim 2 wherein the LF modulated HF coil current is an LF amplitude modulated (AM) HF coil current.

4. A reduced coil current method according to claim 3, wherein the LF modulated HF coil current is an LF double sideband AM HF coil current.

5. A reduced coil current method according to claim 4, wherein generating the LF modulated HF carrier voltage comprises:
generating the HF carrier voltage;
generating a LF signal voltage; and
amplitude modulating the HF carrier voltage by the LF signal voltage, according to a modulation index that is configured to output the LF modulated HF carrier voltage as a double sideband, suppressed carrier AM signal.

6. A reduced coil current method according to claim 1, wherein generating the LF modulated HF carrier voltage comprises:
generating a LF signal voltage;
generating an HF carrier voltage;
frequency modulating the HF carrier voltage by the LF signal voltage and outputting a resultant LF FM modulated HF carrier voltage; and
feeding the LF FM modulated HF carrier voltage to a differentiator which outputs, as a result, a differentiated FM HF voltage as the LF modulated HF carrier voltage.

8. An in tissue electric field generating device, comprising:
a conductive coil;
a controller for applying a low frequency (LF) modulated high frequency (HF) carrier voltage to terminals of the conductive coil so as to force a corresponding LF modulated HF coil current of maximum amplitude (MA) through the conductive coil;
a means for spatially positioning, in a three-dimensional (3D) space, the conductive coil and a living subject into a cooperative 3D spatial relation wherein a target 3D region of the living subject's tissue is within a designated 3D electric field formation region for the conductive coil,
wherein the HF frequency is greater than approximately 8 KHz and the LF frequency is less than approximately 4 KHz, and
wherein the MA, the HF frequency, and a dimension and a geometry of the conductive coil are configured in combination to produce a time-changing magnetic flux density that establishes within the designated 3D electric field formation region an electric field having a magnitude that is at least approximately 100 volts per meter.

9. The in tissue field generating device according to claim 8, wherein the LF modulation is a sinusoid and HF carrier is a sinusoid.

10. The in tissue field generating device according to claim 8, wherein the LF modulated HF coil current is an LF amplitude modulated (AM) HF coil current.

11. The in tissue field generating device according to claim 8, wherein the LF modulated HF coil current is an LF double sideband AM HF coil current.

12. The in tissue field generating device according to claim 8, wherein the LF modulated HF carrier voltage is generated by:
generating the HF carrier voltage;
generating a LF signal voltage; and
amplitude modulating the HF carrier voltage by the LF signal voltage, according to a modulation index that is configured to output the LF modulated HF carrier voltage as a double sideband, suppressed carrier AM signal.

13. The in tissue field generating device according to claim 8, wherein the LF modulated HF carrier voltage is generated by:
generating a LF signal voltage;
generating an HF carrier voltage;
frequency modulating the HF carrier voltage by the LF signal voltage and outputting a resultant LF FM modulated HF carrier voltage; and
feeding the LF FM modulated HF carrier voltage to a differentiator which outputs, as a result, a differentiated FM HF voltage as the LF modulated HF carrier voltage.

14. The in tissue field generating device according to claim 8, further comprising means for LF modulation by a Temporal Interference (TI), including magnetic means of a LF amplitude modulated (AM) HF signal and a HF non modulated carrier.

15. The in tissue field generating device according to claim 8, further comprising means for LF modulation by Temporal Interference (TI) including magnetic means of two HF non modulated carrier of different frequencies.
